(19) United States
(12) Patent Application Publication
(10) Pub. No.: US 2004/0111013 A1
(43) Pub. Date: Jun. 10, 2004

(54) Personal life information managing system

(76) Inventors: Yuan-Yao Shen, Taipei (TW); Ting-Chin Shen, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/314,121

(22) Filed: Dec. 9, 2002

(51) Int. Cl. A61B 5/00

(52) U.S. Cl. 600/300

(57) ABSTRACT

A personal life information managing system that can be mounted on a wireless information transmitter or combined with a fixed detector comprises at least a signal receiving module, a signal processing module, and a data processing module, and can be used in combination with the displaying module, the signal transferring module, and the warning module on the wireless transmitter; wherein upon receiving a personal physiological or biochemical function signal from said signal receiving module, said signal processing module and said data processing module can convert said personal physiological or biochemical function signal into data, and analyze, compare and store said data as well as optionally display said result on said displaying module of said wireless transmitter, emitting a warning signal by said warning signal module to inform the user or output said result to telecommunicate with a distal linkage by said a signal transferring module so as to obtain an effect of monitoring the personal healthy state of the user.

### BACKGROUND OF THE INVENTION

1. Field of the Invention

The invention relates to a personal life information managing system, receiving data from in vivo sensor or in vitro detector and the personal life information managing system that can be mounted on a wireless information transmitter, as a independent device or combined with a fixed detector, and which exhibits a variety of functions such as receiving, analyzing/comparing, storing, and displaying the detected personal life information, as well as revealing, warning and/or telecommunicating indications therein.

2. Description of the Prior Art

As the technology advanced continuously, types and popularity of wireless information transmitter have increased, and as a result, almost everyone has his own personal provision such as mobile phone, personal digital assistant (PDA), pocket personal computer (pocket PC) and the like. Utilization of this situation is accordingly worthy of consideration.

Since technology relating the biochip and physiological or immunological function detecting is progressing rapidly, a number of physiological, biochemical or immunological changes can be monitored in vivo through surface or implant techniques and as well as a private in vitro detector consequently reveal as early as possible whether there is a signature of disease or not. Therefore, how to combine such popular wireless information transmitter with a personal information detector in order to achieve the desired function as instantaneous managing personal life should become now a subject to be studied with emphasis.

Furthermore, as the result of the continuous improvement of biotechnology, in addition to the flourishing development of biochip technology, the advance of microelectronic devices has enabled the detection of personal life information and changes thereof through transdermal or in vivo routes as well as the gastrointestinal tract via oral ingesting. As the result, the processing of this information can be done further in a general manner with a single device.

In view of the above standpoint, the inventor had disclosed in his U.S. Pat. No. 6,428,475, entitled “Mobile Phone Combined Physiological Function Detector” comprised mainly of a mobile phone set capable of receiving detected physiological function signal and a measurer used to touch the human skin and transmit detection signal to the mobile phone set by wire or wireless transmission and revealing the situation of the body function, and in addition, save the utility of machine and material.

The inventor found out the inadequacy of the above mobile phone combined physiological function detector and devoted to improve it. After intensive study, the inventor developed a personal life information managing system and thus accomplished the invention.

### SUMMARY OF THE INVENTION

Accordingly, one object of the invention is to provide a personal life information managing system, characterized in that it can receive transform, analyze/compare, and store the detected personal physiological function signals, and inform the user by displaying the detected personal life information on the screen of the wireless transmitter, such that if there is any physiological, biochemical or immunological abnormality, said system can reveal and emit a warning signal and optionally telecommunicate with a distal hospital to obtain a suggestion for instant treating.

Another object of the invention is to provide a personal life information managing system, characterized in that it can be mounted on a fixed type measurer, and receive, transform by the system, analyze/compare, and store the detected personal physiological function signals, and inform the user by displaying the detected personal life information on the screen of the fixed type measurer, such that if there is any physiological abnormality, said system can reveal and emit a warning signal and optionally telecommunicate with a distal hospital to obtain a suggestion for instant treating.

Still another object of the invention is to provide a personal life information managing system, characterized in that it can receive the detected life function signal emitted by a physiological, biochemical or immunological function detector or a biochip.

Yet still another object of the invention is to provide a personal life information managing system, characterized in that it combines a signal receiving module, a signal procession module, and a data procession module in a signal chip such that it can be used in conjunction with a wireless transmitter, such as a mobile phone, a personal digital assister (PDA), a pocket PC, and the like.

The above-described objects can be achieved by a personal life information managing system according to the invention, which can be mounted on a wireless transmitter or a fixed type measurer and which comprises at least:

a signal receiving module, for receiving measured physiological, biochemical or immunological function signal and transferring said signal to a signal-processing module;

a signal-processing module, for converting said signal from said signal receiving module into data signal and transferring said data signal into a data processing module;

a data processing module, for analyzing and comparing said data signal from said signal processing module, and transferring the output into a displaying module, a signal-transferring module or a warning signal module on a wireless transmitter or fixed type measurer;

wherein upon receiving a life signal from said signal receiving module, said signal processing module and said data processing module can convert into data, and analyze, compare and store said data as well as optionally display said result on the screen a wireless transmitter or a fixed type measurer, emitting a warning signal by said warning signal module or transferring said result by said signal transferring module to a remote linkage so as to obtain an effect of monitoring the personal physiological state of the user.

### BRIEF DESCRIPTION OF THE DRAWINGS

The drawings disclose an illustrative embodiment of the present invention, which serves to exemplify the various advantages and objects thereof, and are as follows:

FIG. 1 is a block diagram showing a personal life information managing system according to the invention mounted on a wireless transmitter;

FIG. 2 is a block diagram illustrating the performing mechanism of a personal life information managing system according to the invention mounted on a wireless transmitter;

FIG. 3 is a block diagram showing a personal life information managing system according to the invention mounted on a fixed type measurer;

FIG. 4 is a block diagram illustrating the performing mechanism of a personal life information managing system according to the invention mounted on a fixed type measurer; and

FIG. 5 is a schematic view illustrating the personal life information managing system according to the invention as an independent device.

### DETAILED DESCRIPTION OF THE PREFERRED EMBODIMENT

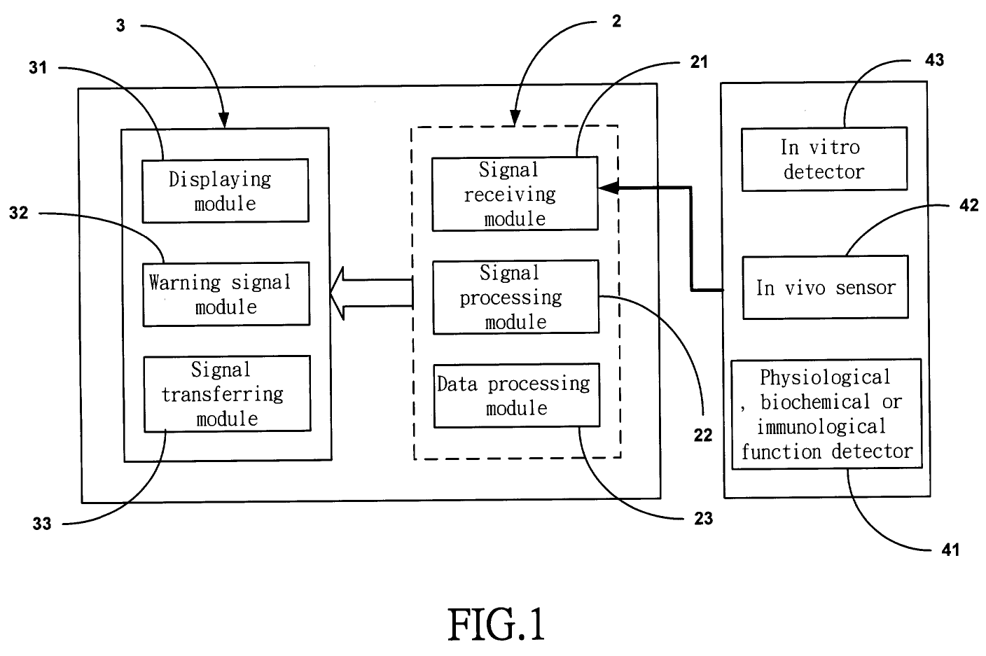

Referring to FIG. 1, showing a block diagram showing a personal life information managing system according to the invention mounted on a wireless transmitter, wherein a personal life information managing system 2 is mounted on a wireless transmitter 3 (e.g., a mobile phone, a personal data assistant, a pocket PC, and the like), and comprises:

a signal receiving module 21, for receiving through wire or wireless manner a measured personal physiological, biochemical or immunological function signal (e.g. blood pressure, pulse, etc.) from a physiological, biochemical or immunological function detector 41, the in vivo sensor 42 or in vitro detector 43 and transferring said signal to a signal processing module 22;

a signal-processing module 22, for converting said personal life function signal from said signal receiving module 21 into data signal and transferring said data signal into a data processing module 23;

a data processing module 23, for analyzing and comparing said data signal from said signal processing module 22, and transferring the output into a displaying module 31, a warning signal module 32 or a signal-transferring module 33 on a wireless transmitter 3;

wherein upon receiving a personal life function signal or in vivo sensor or in vitro detector signal from said signal receiving module 21, said signal processing module 22 and said data processing module 23 can convert said personal life function signal into data, and analyze, compare and store said data as well as optionally display said result said displaying module 31 on a wireless transmitter 3, emitting a warning signal by said warning signal module 32 or transferring said result to a remote linkage by said signal transferring module 33 so as to obtain an effect of monitoring the personal physiological state of the user.

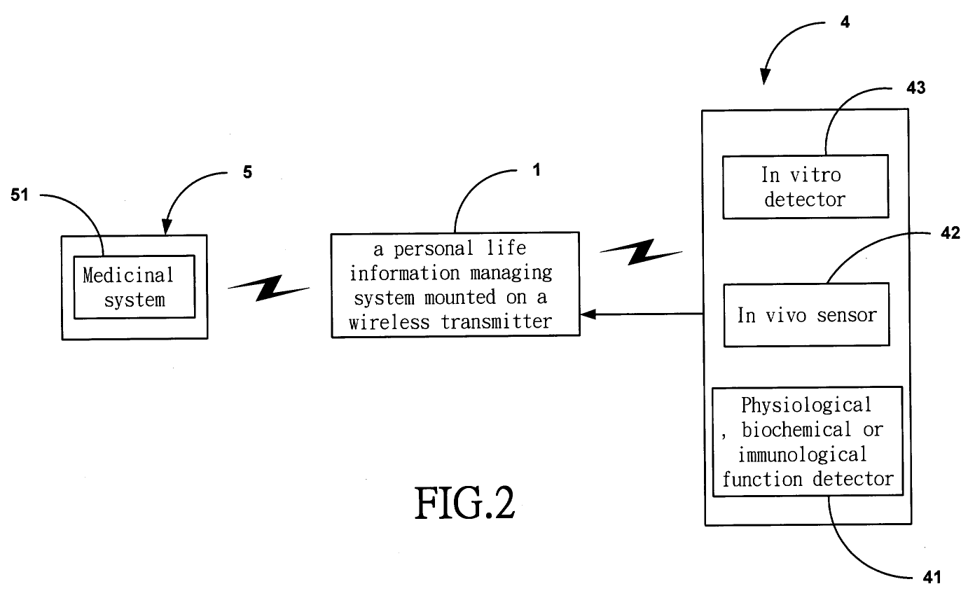

Referring to FIG. 2, a block diagram illustrating the performing mechanism of a personal life information managing system according to the invention mounted on a wireless transmitter, the personal life information managing system 1 mounted on a wireless transmitter is in a position between the medicinal system 51 in a hospital 5 and a user 4 such that, the personal physiological, biochemical or immunological function signal measured by the physiological, biochemical or immunological function detector 41, the in vivo sensor 42 or in vitro detector 43 can be converted and analyzed and thereafter, the result can be transmitted in wireless manner to the medicinal system 51 in the hospital 5. The medicinal system 51 in the hospital 5 can in turn, via this same route, monitor at any time the physiological state of the user 4, inform the emergency treatment, the medicinal instruction, personal medicinal information and the like.

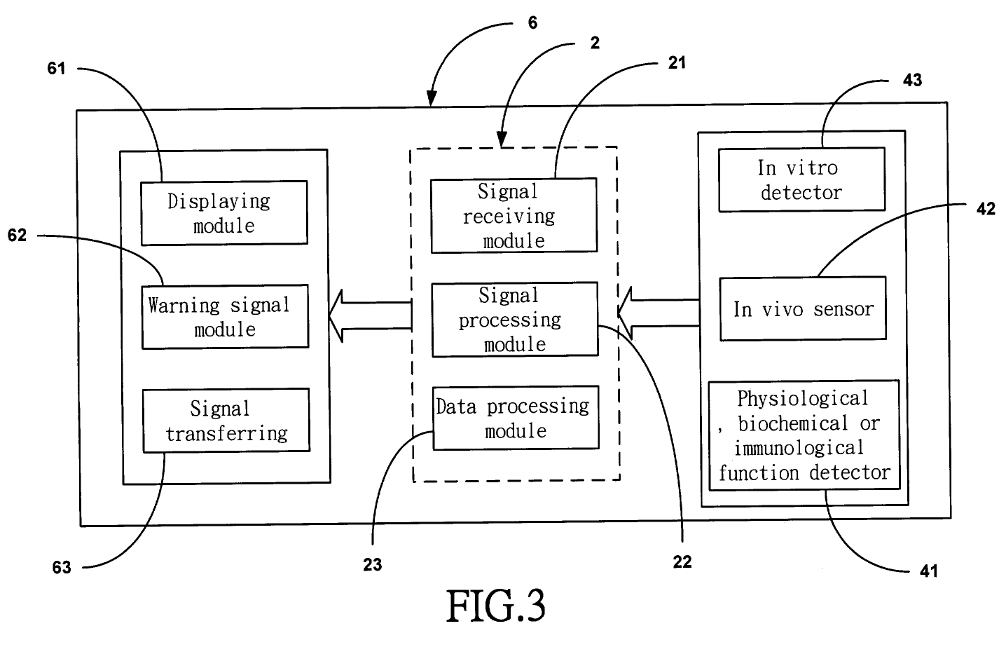

Referring to FIG. 3, a block diagram showing a personal life information managing system according to the invention mounted on a fixed type measurer, a personal life information managing system 2 according to the invention is mounted on a fixed type measurer 6 and comprises:

a signal receiving module 21, for receiving through a wire or wireless manner a measured personal life function signal from a physiological, biochemical or immunological function detector 41 or in vivo sensor 42 or in vitro detector 43 and transferring said signal to a signal processing module 22;

a signal-processing module 22, for converting said personal life function signal from said signal receiving module 21 into data signal and transferring said data signal to a data processing module 23;

a data processing module 23, for analyzing and comparing said data signal from said signal processing module 22, and transferring the output into a displaying module 61, a warning signal module 62 or a signal-transferring module 63 on a fixed type measurer 6;

wherein upon receiving a personal life function signal from said signal receiving module 21, said signal processing module 22 and said data processing module 23 can convert said personal life function signal into data, and analyze, compare and store said data as well as optionally display said result on said displaying module 61 of said fixed type measurer 6, emitting a warning signal by said warning signal module 62 or transferring said result to a remote linkage by said signal transferring module 63 so as to obtain an effect of monitoring the personal physiological state of the user.

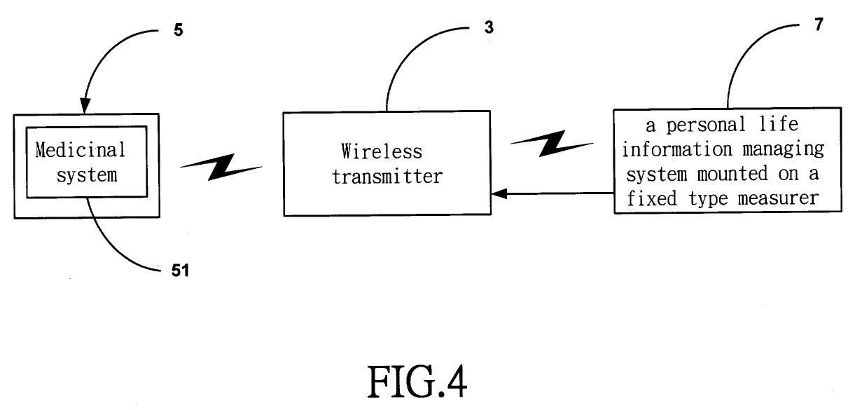

Referring to FIG. 4, a block diagram illustrating the performing mechanism of a personal life information managing system according to the invention mounted on a fixed type measurer, the personal life information managing system 7 mounted on a fixed type measurer can transfer an information signal to a wireless transmitter 3 through wire or wireless manner, and by means of the telecommunicating character of the wireless transmitter 3, communicates the result with the medicinal system 51 in a hospital 5. In turn, the medicinal system 51 in the hospital 5 can, via this same route, monitor at any time the physiological state of the user 4, inform the emergency treatment, the medicinal instruction, personal medicinal information and the like.

The personal life information managing system according to the invention can be fabricated and miniaturized in a chip, and then mounted on various wireless transmitter or fixed type measurer in fixing, inserting or external connection manners.

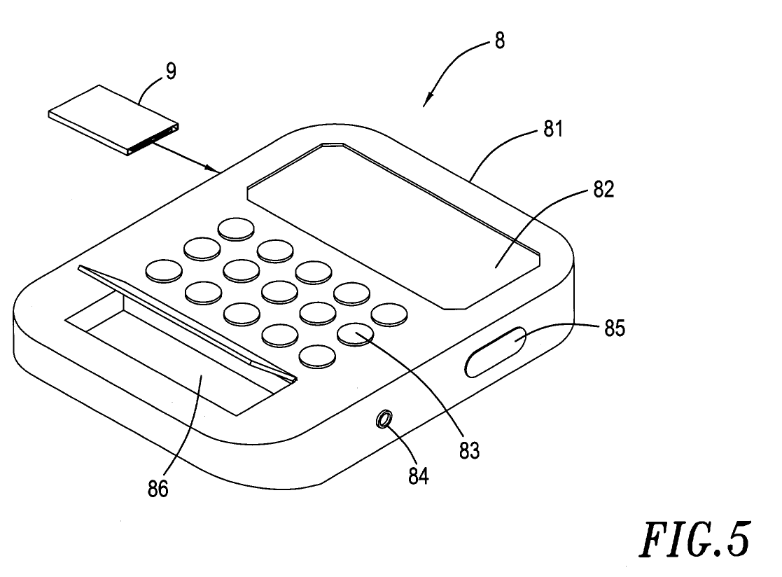

Referring to FIG. 5, a schematic view illustrating the personal life information managing system according to the invention as an independent device, it is apparent that the personal life information managing system according to the invention can be combined with a microprocessor, a display module, warning signal module and signal-transmitting module into an independent device 8. Said device 8 can receive various detection information from biochip detector, physiological function detector, a variety of ingestion-type capsule detector or specimen form human body, and then the device can process, analyze, display, store in memory and warn. Alternatively, the device can transmit the received information via cable or wireless manner through the signal-transmitting module to other communication equipment such as, for example, memory card 91, personal communication apparatus 92 or satellite communication instruments 93, such that a communication with a hospital or medication system 94 can be linked immediately.

The device 8 comprises essentially:

a shell 81, for accommodating the circuit of the system built-in therein, and provided with various functional elements at outside thereof;

a displaying module 82, being provided on said shell 81 for displaying various information and data;

an input keyboard 83, being provided on the top of said shell 81 and for inputting various operation instructions by the user;

a cable transmitting/receiving socket 84, being provided on the outside of shell 81 for connecting with connecting line to input or output various data;

a wireless transmitting/receiving unit 85, being provided on the outside of shell 81 for communicating with other communication equipment via various wireless signals to input or output various data;

a memory card slot, being accommodated in said shell 81 and provided with built-in memory or extended memory card 91 for storing various information and data; and

a specimen cell 86, for holding various specimens from human body (e.g., blood, humoral fluid, saliva, urine, stool, etc.) and detecting them by means of the built-in sensors, thereby inputting data thus obtained into the system.

The device 8 harboring the system of the present invention can be used in conjunction with a digital camera and transmits the image information taken and input by the digital camera together with data analyzed by the system to other communication apparatus.

The personal life information managing system according to the invention has following advantages over the recited patent or other conventional techniques:

1. The personal life information managing system according to the invention can receive personal physiological or biochemical function information that can be converted, analyzed and compared by the system, and thereafter the result can be displayed on the screen of a wireless transmitter. If there is any abnormal personal physiological condition, the system will warn by means of the wireless transmitter, and optionally communicates with the medicinal system of a remote hospital through the wireless transmitter so as to obtain the instruction of instant emergency treatment.

2. The personal life information managing system according to the invention can be mounted on a fixed type measurer, and receive personal physiological or biochemical function information that can be converted, analyzed and compared by the system, and thereafter the result can be displayed on the screen of the fixed type measurer, emits a warning signal by means of the fixed type measurer, and optionally transmits the information in a wire or wireless manner to communicate with the medicinal system of a remote hospital through a wireless transmitter so as to obtain a instant instruction.

3. The personal life information managing system according to the invention can receive the measured personal physiological or biochemical function information emitted from a physiological, physiological, biochemical or immunological function detector, the in vivo sensor or in vitro detector.

4. The personal life information managing system according to the invention can combine a signal receiving module, a signal processing module and a data processing module in a chip such that it can be conveniently mounted on various wireless transmitter, such as mobile phone, personal digital assister (PDA), pocket PC and the like.

5. The personal life information managing system according to the invention can achieve a prophylactic medicinal effect, whereby in the earliest stage of a disease, the condition can be detected and treated as soon as possible and thereby the deterioration of the disease can be prevented and the problem associated with delayed treatment can be avoided.

6. The personal life information managing system according to the invention can eliminate the waste of medicinal resources, whereby in the case of readily treated disease, the patient can get an instruction of an effective treatment just through the linkage of the present system with the medicinal system of a remote hospital.

7. The personal life information managing system according to the invention has a function of keeping a life in a healthy state, and the heavily diseased patient can be-monitored by a medicinal system through the linkage provided by the present system so as to save the medicinal resource and eliminate the laborious burden on the patient.

Many changes and modifications in the above-described embodiment of the invention can, of course, be carried out without departing from the scope thereof. Accordingly, to promote the progress in science and the useful arts, the invention is disclosed and is intended to be limited by the scope of the appended claims.

What is claimed is:

1. A personal life information managing system, that can be mounted on a wireless transmitter or a fixed type measurer and that comprises at least:
a signal-receiving module, for receiving measured physiological, biochemical or immunological function signal from a personal life function detector either in vivo sensor or in vitro detector and transferring said signal to a signal-processing module;
a signal-processing module, for converting said personal physiological or biochemical function signal from said signal receiving module and converting it into a data signal and then transferring said data signal into a data processing module;
a data processing module, for analyzing and comparing said data signal from said signal processing module, and transferring the output into a displaying module, a signal-transferring module or a warning signal module on a wireless transmitter;
wherein upon a personal physiological or biochemical function signal is received by said signal receiving module, said signal processing module and said data processing module can converting said personal physiological, biochemical or immunological function signal into data, and analyze, compare and store said data as well as optionally display said result on the displaying module of said wireless transmitter, emitting a warning signal by said warning signal module or transferring said result by said signal transferring module to a remote linkage in two-way manner so as to obtain an effect of monitoring the personal physiological state of the user.

2. A personal life information managing system as recited in claim 1, characterized in that said personal life information managing system is in a position between the medicinal system in a distal hospital and a user such that, the personal physiological, biochemical or immunological function signal measured by a in vivo sensor or in vitro detector can be converted and analyzed and thereafter, the result can be transmitted in a wireless or wire manner to the medical system in the hospital, whereby the medicinal system in the hospital can in turn, via this same route, monitors at any time the physiological state of the user, informs the emergency treatment, the medicinal instruction, personal medical information and the like.

3. A personal life information managing system as recited in claim 1, characterized in that said wireless transmitter can be any product having a function of wireless communication, such as a mobile phone, a personal digital assistant, a pocket personal computer, and the like.

4. A personal life information managing system as recited in claim 1, characterized in that said signal receiving module can receive through a wireless or wire manner said measured physiological, biochemical or immunological function signal emitted from a personal in vivo sensor or in vitro detector.

5. A personal life information managing system as recited in claim 1, characterized in that said personal life information managing system can be fabricated and miniaturized in a chip, and thereby can be mounted on various wireless transmitter in fixing, inserting or external connection manners.

6. A personal life information managing system, characterized in that it is mounted on a fixed type measurer and comprises:
a signal receiving module, for receiving through a wire or wireless manner a measured personal physiological, biochemical or immunological function signal from a life function detector either a in vivo sensor or in vitro detector and transferring said signal to a signal-processing module;
a signal-processing module, for converting said personal physiological or biochemical function signal from said signal-receiving module into data signal and transferring said data signal to a data processing module;
a data processing module, for analyzing and comparing said data signal from said signal processing module, and transferring the output into a displaying module, a warning signal module or a signal-transferring module on said fixed type measure;
wherein upon a personal physiological or biochemical function signal from signal receiving module, said signal processing module and said data processing module can convert said personal physiological or biochemical function signal into data, and analyze, compare and store said data as well as optionally display said result on the displaying module of said fixed type measure, emitting a warning signal by said warning signal module or output said result in a wire or wireless manner to telecommunicate with a distal linkage by said a signal transferring module so as to obtain an effect of monitoring the personal healthy state of the user.

7. A personal life information managing system as recited in claim 6, characterized in that the information processed by said personal life information managing system can be transmitted in a wireless or wire manner to a wireless transmitter, and by means the character of telecommunication of said wireless transmitter, said analysis result can be communicated to the medicinal system in a distal hospital, whereby the medicinal system in the hospital can in turn, via this same route, monitor at any time the physiological state of the user, inform the emergency treatment, the medicinal instruction, personal medicinal information, and the like.

8. A personal life information managing system as recited in claim 6, characterized in that said personal life information managing system can be fabricated and miniaturized in a chip, and thereby can be mounted on various wireless transmitter or fixed type measurer in fixing, inserting or external connection manners.

9. A personal life information managing system as recited in claim 1, characterized in that said wireless transmitter can be any product having a function of wireless communication, such as, a mobile phone, a personal digital assistant, a pocket personal computer, and the like.

10. A personal life information managing system characterized in that it comprises a personal life information managing system as recited in claim 1 in combination with a microprocessor, a display module, warning signal module and signal-transmitting module into an independent device, wherein said device can receive various detection information from biochip detector, physiological function detector, a variety of ingestion-type capsule detector or specimen form human body, and then the device can process, analyze, display, store in memory and warn; alternatively, the device can transmit the received information via cable or wireless manner through the signal-transmitting module to a distal end.

11. A personal life information managing system as recited in claim 10 characterized in that said device comprises at least:
a shell, for accommodating the circuit of the system built-in therein, and provided with various functional elements at outside thereof;
a displaying module, being provided on said shell for displaying various information and data;
an input keyboard, being provided on the top of said shell and for inputting various operation instructions;
a cable transmitting/receiving socket, being provided on the outside of shell for connecting with connecting line to input or output various data;
a wireless transmitting/receiving unit, being provided on the outside of shell for communicating with other communication equipment via various wireless signals to input or output various data;
a memory card slot, being accommodated in said shell and provided with built-in memory or extended memory card for storing various information and data; and
a specimen cell, for holding various specimens from human body and detecting them by means of the built-in sensors, thereby inputting data thus obtained into the system.

12. A personal life information managing system as recited in claim 10 characterized in that said device can be used in combination with a digital camera, and transmits the image information taken and input by the digital camera together with data analyzed by the system to other communication apparatus.

13. A personal life information managing system as recited in claim 11 characterized in that said wireless transmitting/receiving unit is capable of outputting to a personal communication device or a satellite communication instruments, such that a communication with the medication system of a hospital can be linked subsequently.

14. A personal life information managing system as recited in claim 13 characterized in that said personal communication comprises cell phone, personal digital assistor or pocket personal computer that has wireless transmission function.
